(19) United States
(12) Patent Application Publication
(10) Pub. No.: US 2025/0189478 A1
(43) Pub. Date: Jun. 12, 2025

(54) METHOD FOR MANUFACTURING ELECTRODES

(71) Applicant: IMEC VZW, Leuven (BE)

(72) Inventors: Jelle Fondu, Kessel-Lo (BE); Simone Severi, Leuven (BE)

(21) Appl. No.: 18/969,063

(22) Filed: Dec. 4, 2024

(30) Foreign Application Priority Data
Dec. 8, 2023 (EP) 23215275.1

(51) Int. Cl. G01N 27/327

(52) U.S. Cl. CPC G01N 27/327

(57) ABSTRACT

The present disclosure relates to a method for manufacturing a plurality of electrodes wherein the electrodes comprise noble metal. The method includes providing a substrate having a dielectric layer, forming a conductive layer on the dielectric layer and in physical contact with the top surface of the sacrificial part, and providing a masking layer over the conductive layer. The method further includes patterning the masking layer to expose at least the conductive layer above the sacrificial part thereby defining a plurality of electrodes. The method also includes etching the conductive layer by ion-beam thereby forming the plurality of electrodes, etching at least a portion of the sacrificial part by dry etching, and removing the masking layer. This disclosure also provides an electrode array obtained by the method described herein, and provides an electroactive device for bio-material processing including the electrode array obtained by the method described herein.

### CROSS-REFERENCE TO RELATED APPLICATIONS

The present application is a non-provisional patent application claiming priority to European Patent Application No. 23215275.1, filed on Dec. 8, 2023, the contents of which are hereby incorporated by reference.

### FIELD OF THE DISCLOSURE

The present disclosure is generally related to a method of manufacturing a plurality of electrodes and more specifically to a method of manufacturing a plurality of electrodes comprising noble metal.

### BACKGROUND

Noble metals, such as gold, platinum, and silver, are often used as electrode materials due to their high conductivity, chemical stability, and resistance to oxidation and corrosion. These properties make them useful in a variety of applications, including electrochemical actuator, batteries and fuel cells.

Electrical leakage is an unintended flow of electrical current between the electrodes, leading to a loss of efficiency and potential loss of accuracy in the device. Electrical leakage can be caused by a variety of factors, including the presence of impurities or defects in the electrode material, or the formation of conductive pathways between the electrodes due to the accumulation of ions or other charged species. One issue that can arise when using noble metal electrodes is electrical leakage due to the re-sputtering of the noble metal particles during manufacturing methods for electrode array in low pitch.

Etching methods for noble metals, such as gold, platinum, and silver, may have drawbacks, such as the re-sputtering of metal particles. During the etching process, high-energy ions may cause the metal particles to be ejected from the surface, leading to re-sputtering. This may result in the formation of conductive pathways between the electrodes, increasing the risk of electrical leakage.

When the distance between electrodes gets smaller during manufacturing, the risk of electrical leakage may increase. This is because the smaller distance between the electrodes can make it easier for conductive pathways to form, allowing current to flow between the electrodes.

### SUMMARY

The disclosure is set out in the claims.

It is an objective of the present disclosure to provide reliable methods for making electrodes comprising noble metal with low leakage between the electrodes. In the present disclosure, low leakage may be that the resistance between electrodes to be at least 1e8 ohm. With the methods in the present disclosure, the distance between the two neighboring electrodes can be usefully scaled to not larger than 500 nm, or not larger than 300 nm, or not larger than 200 nm, or not larger than 150 nm. To minimize the risk of electrical leakage in devices with such small pitch sizes, the manufacture of the electrodes may be carefully controlled to minimize or prevent the buildup of conductive particles (e.g., metal particles) between the electrodes.

Reducing the effect of re-sputtering may be useful for eliminating electrical leakage, such as when electrodes are used in bio-sensing and bio-actuating in liquid environments. To minimize the risk of electrical leakage, the etching process and use methods that reduce the impact of re-sputtering may be used.

The present disclosure uses (e.g., economically efficient) methods provided for making an electrode array having a pitch not larger than 500 nm, or not larger than 300 nm, or not larger than 200 nm, or not larger than 150 nm. Semiconductor device pitch may be provided (e.g., defined) as the combined length of a (e.g., single) printed feature along with the adjacent space. In the present disclosure, this is (e.g., equivalent to) the distance measured from the centerline of a space between two neighboring electrodes (or electrode columns) to the centerline of the next space adjacent to the next pair of electrodes. This may be the distance between the imaginary line that runs through the centers of spaces situated between each pair of neighboring electrodes or electrode columns.

The present disclosure provides (e.g., reliable) methods for making electrodes wherein the dimensions of the electrodes can be (e.g., well) controlled.

The present disclosure provides that at least a thousand electrodes or at least a million electrodes can be manufactured.

In some embodiments, it may be useful to include the further step of reactive ion etching in the method to further disable the leakage path formed by re-sputtered noble metal particles between the electrodes.

In some embodiments, it may be useful for the sacrificial part to comprise a protruded structure of the dielectric layer. These methods may prevent the buildup of conductive particles, e.g., re-sputtered noble metal particles, between the electrodes during the ion-beam etching step.

### BRIEF DESCRIPTION OF THE FIGURES

The above, as well as additional, features will be better understood through the following illustrative and non-limiting detailed description of example embodiments, with reference to the appended drawings.

The disclosure will be further elucidated with the following description and the appended figures. Various exemplary embodiments are described herein with reference to the following figures, wherein like numeral denotes like entities. The figures described are schematic and are non-limiting. Further, any reference signs in the claims shall not be construed as limiting the scope of the present disclosure. Still further, in the different figures, the same reference signs refer to the same or analogous elements.

The terms “over” and “above” are used for position indication of layers and may not necessarily describe a direct contact of the layers. The terms used are interchangeable under appropriate circumstances. The term “on” is used for position indication of layers and describing a (e.g., direct) contact of the layers.

The term “top surface” is used as a reference for a (e.g., certain) surface. The “top surface” can be a bottom surface in figures under appropriate circumstances, for example when the surface/stack is turned around.

FIGS. 1a, 1b, 1c, 1d, 1e, 1f, 1g, and 1h show an example schematic illustration of a cross section of a plurality of stacks resulting from the steps of the process in a first example.

FIGS. 2a, 2b, 2c, 2d, 2e, 2f, 2g, and 2h show an example schematic illustration of a cross section of a plurality of stacks resulting from the steps of the process in a second example.

FIG. 3 shows an example top-down schematic illustration of an electrode array resulting from the steps of the process in the present disclosure.

FIG. 4 shows an exemplary flow chart of the main steps of an embodiment of the manufacture method in the present disclosure.

All the figures are schematic, not necessarily to scale, and generally show parts which elucidate example embodiments, wherein other parts may be omitted or (e.g., merely) suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

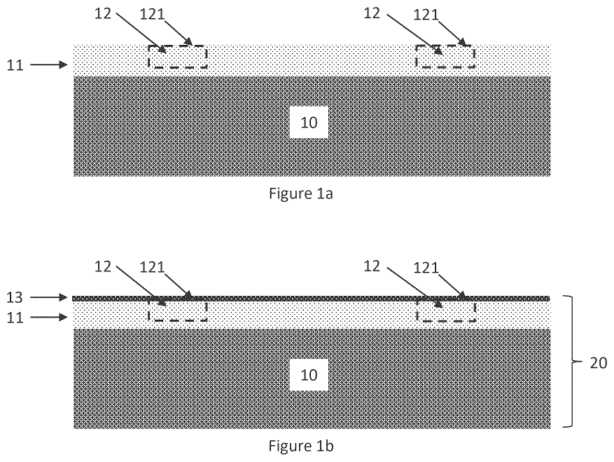

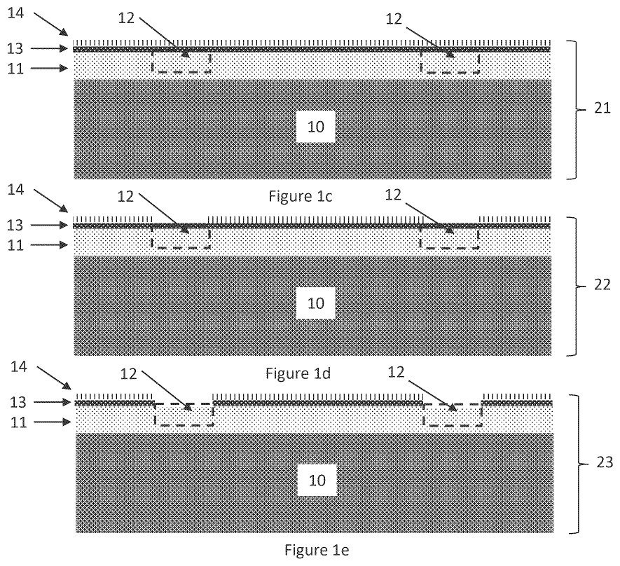

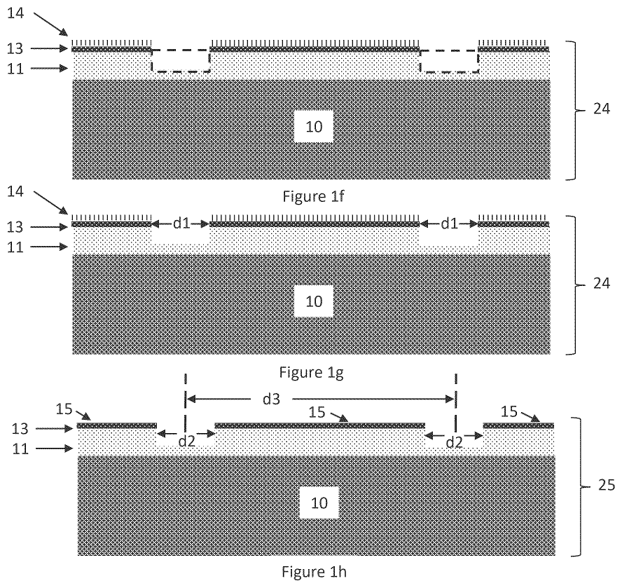

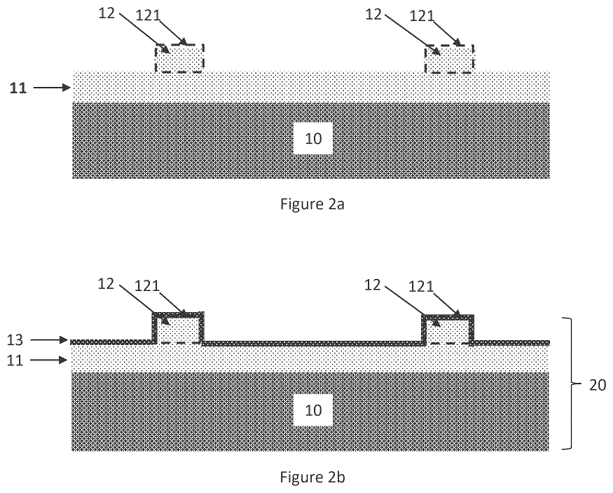

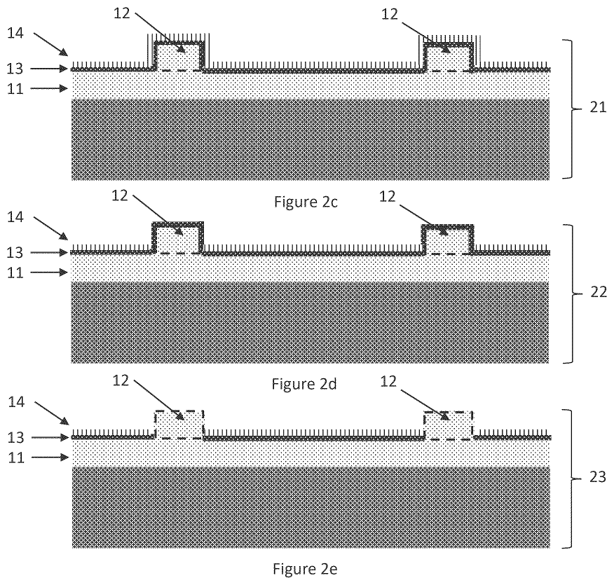

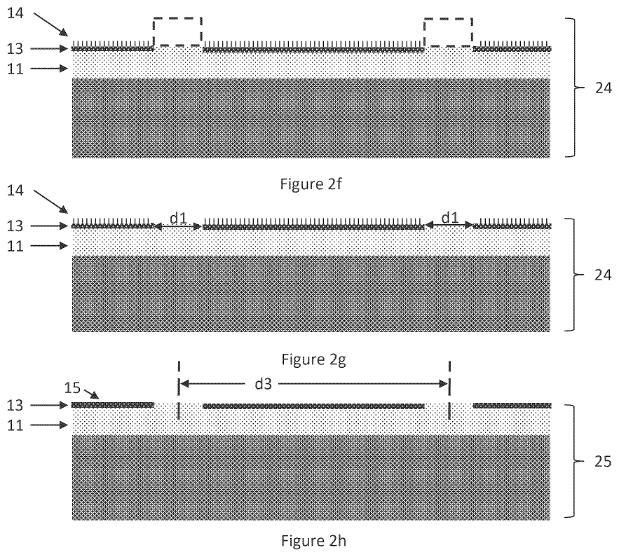

In one implementation, the first aspect of the present disclosure relates to the manufacture of a plurality of electrodes (15) wherein the electrodes (15) comprise noble metal as exemplified in FIGS. 1a to 1h and FIGS. 2a to 2h.

The method may include the steps of: (a) providing a substrate (10) having a dielectric layer (11) in physical contact therewith, wherein the dielectric layer (11) comprises at least a sacrificial part (12), wherein the sacrificial part (12) comprises a top surface (121); (b) forming a conductive layer (13) on the dielectric layer (11) and in physical contact with the top surface (121) of the sacrificial part (12), wherein the conductive layer (13) comprises the noble metal, thereafter; (c) providing a masking layer (14) over the conductive layer (13), thereafter; (d) patterning the masking layer (14) to expose at least the conductive layer (13) above the sacrificial part (12) thereby defining a plurality of electrodes (15) wherein the smallest distance (d1) between the plurality of electrodes (15) is not larger than 500 nm, or not larger than 300 nm, or not larger than 200 nm, or not larger than 150 nm, thereafter; (e) etching the conductive layer (13) by ion-beam thereby forming the plurality of electrodes (15) comprising the noble metal, thereafter; (f) etching at least a portion of the sacrificial part (12) by dry etching such that the resistance at the smallest distance (d1) between the plurality of electrodes (15) is at least 1e8 ohm; and/or (g) removing the masking layer (14).

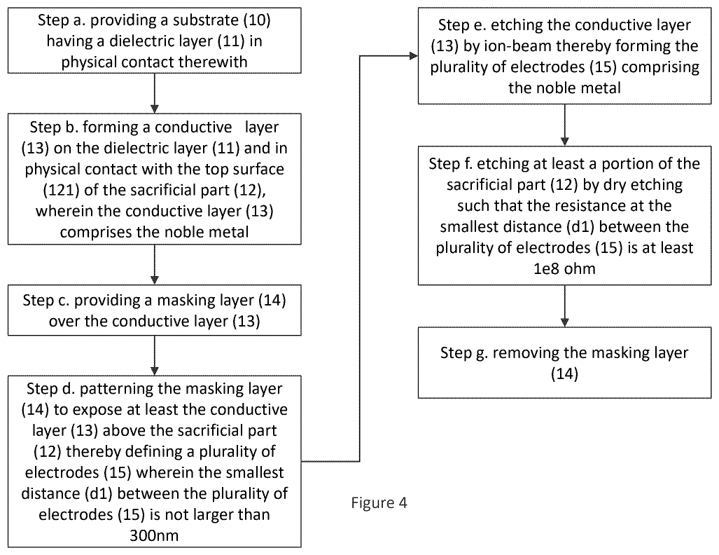

FIG. 4 shows an exemplary flow chart of the main steps of an embodiment of the manufacture method in the present disclosure.

In some embodiments, the masking layer (14) is in (e.g., physical) contact with the conductive layer (13) in step (c).

In some embodiments, step (g) of removing the masking layer (14) is (e.g., directly) after the step (f) of etching at least a portion of the sacrificial part (12) by dry etching.

In some embodiments, step (g) of removing the masking layer (14) is after the step (e) of etching the conductive layer (13) by ion-beam thereby forming the plurality of electrodes (15) and before step (f) of etching at least a portion of the sacrificial part (12) by dry etching, wherein the dry etching is a non-ion-beam etching.

In some embodiments, the step (d) of patterning the masking layer (14) to expose at least part of the conductive layer (13) above the sacrificial part (12) provides (e.g., defines) the edge of the plurality of electrodes (15) such that the smallest distance (d1) between the plurality of electrodes (15) is designed to be not larger than 500 nm, or not larger than 300 nm, or not larger than 200 nm, or not larger than 150 nm.

In some embodiments, the masking layer (14) is formed as a conformal layer over the conductive layer (13).

In some embodiments, the sacrificial part (12) is the same material as in the dielectric layer (11) thus being part of the dielectric layer (11).

In some embodiments, the conductive layer (13) above and corresponding to the sacrificial part (12) is etched (e.g., substantially completely) by ion-beam thereby forming the plurality of electrodes (15) in step (e).

The present disclosure concerns the manufacture of electrodes comprising at least one (e.g., type of) noble metal. An electrode is a conductor through which electric current can pass. Noble metals, such as gold, platinum, and silver, may be used as electrode materials due to their high conductivity, chemical stability, and resistance to oxidation and corrosion, even in liquid environment. These properties make them useful in a variety of applications, including electrochemical actuator, batteries, and fuel cells.

In some embodiments, the noble metal is selected from platinum (Pt), gold (Au), and/or silver (Ag). In an example embodiment, the noble metal is platinum.

As shown in the Figures, the substrate (10) is a base material that supports the formation of other layers on top of it. In this application, the substrate is, in one example, composed of a semiconductor material, which is a type of material that can conduct electricity under certain conditions. For example, the substrate may be a circular wafer or a rectangular wafer die. In some embodiments, the substrate (10) comprises Silicon (Si), which is a material that may be used for integrated circuits and microelectronics devices. Silicon has high purity, low cost, and excellent electrical and thermal properties. The substrate can comprise other semiconductor materials such as Gallium arsenide (GaAs), Sapphire (Al₂O₃), Germanium (Ge).

In some embodiments, the substrate (10) comprises (e.g., consists of) semiconductor material. In some embodiment, the substrate (10) comprises (e.g., consists of) silicon.

In some embodiments, the substrate (10) comprises a stack of semiconductor layers. In some embodiments, the substrate (10) comprises a conductive layer for forming buried conductive connections between electrodes. In some embodiments, the substrate (10) comprises conductive layers and dielectric layers configured for forming switches or other control logic.

The substrate (10) has a dielectric layer (11) in (e.g., physical) contact therewith.

A dielectric layer (11) is a layer of material that has a high electrical resistance and can store electric charge. Dielectrics are often used as insulators or capacitors in electronic devices. The dielectric layer (11) may be any material that can be etched or dissolved by a chemical solution, such as silicon (di)oxide (e.g., SiO₂), silicon nitride (Si₃N₄), or polyimide. The dielectric layer (11) may have a thickness ranging from 10 nm to 10 μm. The dielectric layer (11) comprises a sacrificial part (12) that has a top surface (121). In some embodiments, the properties of the dielectric layer (11), such as thickness or material, are selected and designed such that the resistance between the electrodes (15) at the smallest distance can reach at least 1e8 ohm.

The dielectric layer (11) may either be an (e.g., integral) part of the substrate (10) or a separate layer deposited on one of the (e.g., largest) surfaces of the substrate (10) by any suitable method, such as chemical vapor deposition (CVD), physical vapor deposition (PVD), atomic layer deposition (ALD), or spin coating. In either case, the dielectric layer (11) is (e.g., always) in direct contact with the substrate. The dielectric layer (11) may have a uniform or non-uniform composition and thickness across the substrate (10). The dielectric layer (11) may also have different properties, such as refractive index, dielectric constant, or stress, depending on the application.

In some embodiments, the dielectric layer comprises (e.g., consist of) silicon oxide (e.g., SiₓO_{y}, such as SiO₂) and/or silicon nitride (e.g., SiₓN_{y}, such as Si₃N₄).

In some embodiments, the silicon nitride layer is deposited by chemical vapour deposition (CVD), e.g., plasma-enhanced CVD (PECVD) or low-pressure CVD (LPCVD).

In some embodiments, the silicon oxide is deposited by chemical vapour deposition (e.g., PECVD or LPCVD) or is formed by thermal oxidation of the Si substrate.

The sacrificial part (12) is a portion of the dielectric layer (11) that will be removed in a later step to further enhance the separation between electrodes (15).

The conductive layer (13) is a layer of electrically conductive material that is deposited on the dielectric layer (11) by any suitable method, such as sputtering, evaporation, electroplating, and/or electroless plating. The conductive layer (13) may have a uniform or non-uniform composition and thickness across the dielectric layer (11). The conductive layer (13) comprises at least one type of noble metal. The conductive layer (13) may comprise a single material, e.g., a noble metal or alloy thereof, or a combination of materials, such as with different type of noble metals, alloys. In the present disclosure, the conductive layer (13) may comprise a noble metal, such as platinum (Pt), gold (Au), silver (Ag), or palladium (Pd), which has high conductivity, low reactivity, and good compatibility with organic materials.

The top surface of the substrate (10) can be the top surface of a silicon wafer which is a substantially smooth and flat surface that serves as a base for, e.g., depositing various layers of materials. In an embodiment, the top surface of the substrate of a wafer may also have different patterns, such as trenches, holes, or pillars, that are formed by lithography and etching processes.

In some embodiments, the top surface of the substrate (10) is planarized by chemical mechanical polishing (CMP) before step (b), which involves forming a conductive layer (13) on the dielectric layer (11) and in physical contact with the top surface (121) of the sacrificial part (12), wherein the conductive layer (13) comprises a noble metal.

In some embodiments, the top surface of the conductive layer (13) is planarized by chemical mechanical polishing (CMP) before step (c), which involves providing a masking layer (14) in physical contact with the conductive layer (13).

In some embodiments, the masking layer comprises (e.g., consist of) Silicon Oxide (e.g., SiₓO_{y}, such as SiO₂), silicon nitride (e.g., SiₓN_{y}, such as Si₃N₄), Titanium (Ti), Titanium nitride (TiN) and/or diamond like carbon (DLC).

In some embodiments, the silicon nitride layer is deposited by chemical vapour deposition (e.g., PECVD or LPCVD).

In some embodiments, the silicon oxide is deposited by chemical vapour deposition (e.g., PECVD or LPCVD).

In some embodiments, the Ti or TiN is deposited by chemical vapour deposition (e.g., PECVD or LPCVD) or atomic layer deposition (ALD).

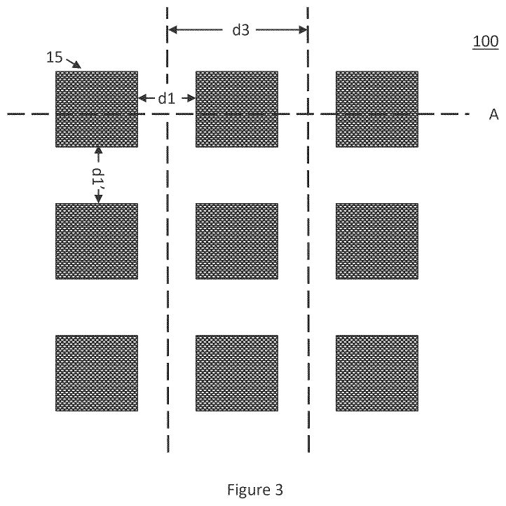

The second aspect of the present disclosure relates to an electrode array (100), as shown in FIG. 3, obtained by the method described in the present disclosure. FIG. 1h or FIG. 2h in the first and second examples of the first aspect can be an exploded (e.g., zoomed-in) cross section along the indication line A in FIG. 3.

In some embodiments, the array has a pitch distance (d1) of not larger than 500 nm, or not larger than 300 nm, or not larger than 200 nm, or not larger than 150 nm. The pitch distance can be measured between a pair of neighboring electrodes, e.g., in the direction of indication line A (d1) or in the direction perpendicular to the indication line A (d1′). In an embodiment, the distance between the pair of neighboring in the direction of indication line A (d1) is substantially equal to the distance between the pair of neighboring in the direction perpendicular to indication line A (d1′).

In some embodiments, the array comprises at least a thousand electrodes or a million electrodes.

The third aspect of the present disclosure relates to an electroactive device for bio-material processing comprising the electrode array obtained by the method described in the present disclosure, and a reservoir for containing the bio-material in an electrolyte during (e.g., the device's) operation, wherein the electrode array is arranged such that the electrode array is exposed to the electrolyte during (e.g., the device's) operation.

In some embodiments, the bio-material is biomolecules such as proteins, carbohydrates, lipids, and nucleic acids (e.g., DNA, RNA).

In some embodiments, the bio-material is biologic tissues such as cells and organoids.

In some embodiments, the electroactive device is configured for DNA or RNA synthesis on electrodes.

### Example 1

As shown in FIG. 1a, the substrate (10) is provided having a dielectric layer (11). Sacrificial part (12) is indicated in the dashed square. The sacrificial part (12) is an (e.g., integral) part of the dielectric layer (11).

The top surface of the dielectric layer (11) is planar and the top surface (121) of the sacrificial part (12) is coplanar with a (e.g., directly) surrounding top surface of the dielectric layer (11).

In some embodiments, the top surface of the dielectric layer (11) is planarized by Chemical Mechanical Polishing (CMP) before step (b) of forming a conductive layer (13) on the dielectric layer (11) and in physical contact with the top surface (121) of the sacrificial part (12), wherein the conductive layer (13) comprises a noble metal.

As shown in FIG. 1b, the conductive layer (13) is deposited directly on the dielectric layer (11). Thus, a stack (20) comprising the substrate (10), the dielectric layer (11), and the conductive layer (13) is formed after step (b) and before step (c).

In some embodiments, the conductive layer (13) (e.g., completely) covers the top surface (121) of the sacrificial part (12), as shown in FIG. 1b. The conductive layer (13) may have a thickness ranging from 10 nm to 100 nm, or from 10 nm to 20 nm.

As shown in FIG. 1c, the masking layer (14) is deposited (e.g., directly) on the conductive layer. Thus, a stack (21) comprising the substrate (10), the dielectric layer (11), the conductive layer (13) and the masking layer is formed after step (c) and before step (d).

The masking layer (14) is provided in (e.g., physical) contact with the conductive layer (13), as shown in FIG. 1c. In an embodiment, the dielectric layer, serving as a masking layer (14), is deposited over the conductive layer (13). The masking layer (14) (e.g., completely) covers the conductive layer (13) in the region corresponding to the sacrificial part (12). In an embodiment, the masking layer (14) may be made of a composite material comprising SiO₂, Si₃N₄, Ti and/or TiN. The masking layer (14) may be deposited by any suitable technique, such as chemical vapor deposition (CVD), atomic layer deposition (ALD), physical vapor deposition (PVD), and/or sputtering. The masking layer (14) can have a thickness ranging from 10 nm to 10 μm, or 5 nm to 1 μm.

As shown in FIG. 1d, the masking layer (14) is patterned. The patterning of the masking layer (14) may expose at least part of the conductive layer to be etched, thereby providing (e.g., defining) the boundaries of the electrodes. A stack (22) comprising the substrate (10), the dielectric layer (11), the conductive layer (13) and a patterned masking layer is formed after step (d) and before step (e).

The masking layer (14) is patterned to expose at least part of the conductive layer (13) in the regions corresponding to the sacrificial part (12). In an embodiment, the masking layer (14) is patterned to expose at least 70% of the conductive layer (13) in the regions corresponding to the sacrificial part (12). In an embodiment, the masking layer (14) is patterned to expose (e.g., completely) the conductive layer (13) in the regions corresponding to the sacrificial part (12). The patterning can be performed by any suitable technique, such as photolithography.

As shown in FIG. 1e, the exposed part of the conductive layer (13) is etched by ion-beam etching. The ion-beam etching is a directional process that can (e.g., effectively) remove the conductive material. In this example, ion-beam etching uses Ar, Xe, Ne, or Kr (e.g., the noble inert gases) which may provide physical etching or sputtering and provides, among other processes, etching of noble metals such as Au, Pt, Pd which are non-reactive materials and hence do not respond to reactive plasma or chemical etching. The ion-beam etching can have an etching rate ranging from 1 nm/min to 100 nm/min depending on the ion source, the voltage, and the angle of incidence. The ion-beam etching can also be controlled by adjusting the duration and the area of exposure. The ion-beam etching removes the conductive layer (13) to form electrodes (15) on the dielectric layer (11) in the regions not corresponding to the sacrificial part (12). A stack (23) comprising the substrate (10), the dielectric layer (11), a patterned conductive layer (13), e.g., the electrodes (15), and a patterned masking layer (14) is formed after the step (e) and before step (f).

The dielectric layer serves as a masking layer to protect the conductive layer (13) from being removed in the regions not corresponding to the sacrificial part (12). However, the ion-beam etching will consume part of the masking layer (14). Thus, the masking layer (14) shall have a thickness and characteristics such that it is not (e.g., completely) removed before the conductive layer in the regions corresponding to the sacrificial part (12) is removed. In an embodiment, the masking layer shall have at least a thickness of 25% of the thickness before ion-beam etching. This provides that the electrodes (15) are (e.g., well defined and) aligned with the opening (13). Alternatively, the masking layer (14) can have a higher etching selectivity than the conductive layer (13), e.g., a lower etching rate, to withstand the ion-beam etching longer than the conductive layer (13). In an embodiment, the conductive layer corresponding to the sacrificial part (12) is (e.g., completely) removed, providing that no electrodes may be formed on top of the sacrificial part (12).

As a side effect of the ion-beam etching, some noble metal particles are generated from the conductive layer (13) and re-sputtered in the space between the electrodes (15), e.g., above or on the surface of the sacrificial part (12). These noble metal particles can impact the electrical characteristics and performance of the device.

Sometimes a cleaning process may be performed to remove the re-sputtered noble metal particles from the space between the electrodes (15) by using a solvent, such as acetone, isopropanol, or ethanol, and applying ultrasonic waves to dissolve and dislodge the noble metal particles; however, the cleaning process using a solvent and ultrasonic wave may have some drawbacks, such as the solvent can penetrate into the space between the electrodes (15) and the dielectric layer (11), which can cause swelling or cracking of the dielectric layer (11). Another drawback may be the solvent reacting with the dielectric layer (11) or the electrodes (15), which can degrade their properties or cause corrosion, and furthermore, the ultrasonic waves can generate mechanical stress or vibration on the electrodes (15), which can damage or detach them from the dielectric layer (11). Therefore, some cleaning processes using a solvent and ultrasonic wave may compromise the quality and reliability of the device. Furthermore, another drawback may be that noble metal, such as Pt, is not removable by (e.g., standard) cleaning solvents; however, the nobel metal may be etched by aggressive solvent such as aqua regia.

As shown in FIG. 1f, the sacrificial part (12) is etched by dry etching, e.g., reactive ion etching. The reactive ion etching can (e.g., selectively) remove the material of the sacrificial part (12) without damaging the electrodes (15). In some embodiments, the electrodes act as hard mask for reactive ion etching the sacrificial part (12).

It is better in this example if at least part of the step (f) of etching is provided by a non-ion-beam dry etch so that (e.g., further) re-sputtering of metal particles is prevented. In an embodiment, part of the sacrificial part (12) is etched by ion-beam etching. Because of the non-ion-beam dry etch, the density of metal particles close to the edge of the electrode is significantly reduced thus increasing the resistance between the electrodes.

Thus, the resistance at the smallest distance (d1) between the plurality of electrodes (15), as indicated in FIG. 1g, is at least 1e8 ohm.

The dry etching may have an etching rate ranging from 10 nm/min to 1000 nm/min depending on the material of the sacrificial part (12), the etching gas, and the etching parameters. The dry etching may also be controlled by adjusting the duration and the area of exposure. The dry etching removes at least a portion of the sacrificial part (12) to form a cavity that has a depth ranging from 10 nm to 200 nm.

In some embodiments, after the sacrificial part is etched, the dielectric layer (11) comprises recessed regions in the dielectric layer (11), corresponding to the removed sacrificial part, is obtained between the plurality of electrodes (15).

A stack (24) comprising the substrate (10), the dielectric layer (11) with recessed regions, a patterned conductive layer (13), e.g., the electrodes (15), and a patterned masking layer (14) is formed after the step (f) and before step (g).

In some embodiments, as a last step, the masking layer (14) is removed by etching methods. A stack (25) comprising the substrate (10), the dielectric layer (11) with recessed region and a patterned conductive layer (13), e.g., the electrodes (15) is formed after step (f).

In some alternative embodiments, the masking layer (14) can be removed before the reactive ion etching of at least part of the sacrificial part (12).

As indicated in FIG. 1g, the distance (d1) between the electrodes is provided (e.g., defined) in the step (d) of patterning the masking layer (14) to expose at least part of the conductive layer (13) above the sacrificial part (12). In an embodiment, the distance (d2) of the recessed region in the dielectric layer between the electrodes is substantively equal to the distance (d1) between the electrodes. Because, in some embodiments, the electrodes act as hard mask for reactive ion etching the sacrificial part (12). In an embodiment, the distance (d2) of the recessed region in the dielectric layer between the electrodes within 10% margin of the distance (d1) between the electrodes. In an example embodiment, the distance (d2) of the recessed region in the dielectric layer between the electrodes is within 5% margin of the distance (d1) between the electrodes.

The pitch (d3), as indicated in FIG. 1h, is provided (e.g., defined) after step (e) of etching the conductive layer (13) by ion-beam thereby forming the plurality of electrodes (15).

The distance (d1) between the electrodes can be the smallest distance between any two electrodes of the plurality of electrodes. The resistance of the two electrodes which has the smallest distance can be less than 1e8 ohm after dry etching step (f).

To measure the resistance between the electrodes, the probes of the multimeter or the ohmmeter can be connected to the two electrodes whose resistance is to be measured.

### Example 2

As shown in FIG. 2a, the substrate (10) is provided having a dielectric layer (11). Sacrificial part (12) is indicated in the dashed square. The sacrificial part (12) is an (e.g., integral) part of the dielectric layer (11).

The sacrificial part (12) is a protruded structure of the dielectric layer (11).

As shown in FIG. 2b, the conductive layer (13) is deposited (e.g., directly) on the dielectric layer (11). Thus, a stack (20) comprising the substrate (10), the dielectric layer (11), the conductive layer (13) is formed after step (b) and before step (c).

In some embodiments, the conductive layer (13) (e.g., completely) covers the top surface (121) of the sacrificial part (12), as shown in FIG. 2b. The conductive layer (13) may have a thickness ranging from 10 nm to 100 nm, or from 10 nm to 20 nm.

As shown in FIG. 2c, the masking layer (14) is deposited (e.g., directly) on the conductive layer. Thus, a stack (21) comprising the substrate (10), the dielectric layer (11), the conductive layer (13) and masking layer is formed after step (c) and before step (d).

The masking layer (14) is provided in physical contact with the conductive layer (13), as shown in FIG. 2c. In an embodiment, a dielectric layer, serving as the masking layer (14), is deposited over the conductive layer (13). The masking layer (14) (e.g., completely) covers the conductive layer (13) in the region corresponding to the sacrificial part (12). In an embodiment, the masking layer (14) can be made of a composite material comprising SiO₂, Si₃N₄, Ti and/or TiN. The masking layer (14) can be deposited by any suitable technique, such as chemical vapor deposition (CVD), atomic layer deposition (ALD), physical vapor deposition (PVD), and/or sputtering. The masking layer (14) can have a thickness ranging from 10 nm to 10 μm, or 5 nm to 1 μm.

As shown in FIG. 2d, the masking layer (14) is patterned. The patterning of the masking layer (14) would expose at least part of conductive layer to be etched thereby defining the boundary of the electrodes. A stack (22) comprising the substrate (10), the dielectric layer (11), the conductive layer (13) and a patterned masking layer is formed after step (d) and before step (e).

The masking layer (14) is patterned to expose at least part of the conductive layer (13) above the protruded structure. In an embodiment, the masking layer (14) is patterned to expose at least 70% of the conductive layer (13) in the regions corresponding to the sacrificial part (12). In an embodiment, the masking layer (14) is patterned to expose (e.g., completely) the conductive layer (13) in the regions corresponding to the sacrificial part (12). The patterning can be performed by any suitable technique, such as photolithography. In an embodiment, the masking layer (14) is patterned to expose (e.g., completely) the conductive layer (13) in the regions corresponding to the sacrificial part (12) and a portion of the masking layer (14) in the neighboring regions of the regions corresponding to the sacrificial part (12).

As shown in FIG. 2e, the exposed part of the conductive layer (13) is etched by ion-beam etching. The ion-beam etching is a directional process that can effectively remove the conductive material. In this example, ion-beam etching uses Ar, Xe, Ne or Kr (e.g., the noble inert gases) to enable physical etching or sputtering and allows, among other processes, etching of noble metals such as Au, Pt, Pd which are non-reactive materials and hence do not respond to reactive plasma or chemical etching. The ion-beam etching can have an etching rate ranging from 1 nm/min to 100 nm/min depending on the ion source, the voltage, and the angle of incidence. The ion-beam etching can also be controlled by adjusting the duration and the area of exposure. The ion-beam etching removes the conductive layer (13) to form electrodes (15) on the dielectric layer (11) in the regions not corresponding to the sacrificial part (12). A stack (23) comprising the substrate (10), the dielectric layer (11), a patterned conductive layer (13), e.g., the electrodes (15), and a patterned masking layer (14) is formed after step (e) and before step (f).

The masking layer (14) protects the conductive layer (13) from being removed in the regions not corresponding to the sacrificial part (12). However, the ion-beam etching may consume the masking layer (14). Thus, the masking layer (14) may have a thickness and characteristics such that it is not (e.g., completely) removed before the conductive layer in the regions corresponding to the sacrificial part (12) is removed. In an embodiment, the masking layer may have at least a thickness of 25% of the thickness before ion-beam etching. This can ensure that the electrodes (15) are (e.g., well defined and) aligned with the opening (13). Alternatively, the masking layer (14) can have a higher etching selectivity than the conductive layer (13), e.g., a lower etching rate, to withstand the ion-beam etching longer than the conductive layer (13). In an embodiment, the conductive layer corresponding to the sacrificial part (12) is (e.g., completely) removed thus no electrode will be formed on top of the sacrificial part (12).

As a side effect of the ion-beam etching, some noble metal particles are generated from the conductive layer (13) and re-sputtered in the space between the electrodes (15), e.g., above or on the surface of the sacrificial part (12). These noble metal particles can affect the electrical characteristics and performance of the device. Conventionally, a cleaning process may be performed to remove the re-sputtered noble metal particles from the space between the electrodes (15) by using a solvent, such as acetone, isopropanol, or ethanol, and applying ultrasonic waves to dissolve and dislodge the noble metal particles. However, the cleaning process using a solvent and ultrasonic wave can have some drawbacks. For example, a drawback may be that the solvent can penetrate into the space between the electrodes (15) and the dielectric layer (11), which can cause swelling or cracking of the dielectric layer (11). Moreover, another drawback is that the solvent can also react with the dielectric layer (11) or the electrodes (15), which can degrade their properties or cause corrosion. Furthermore, a drawback is the ultrasonic waves can generate mechanical stress or vibration on the electrodes (15), which can damage or detach them from the dielectric layer (11). Therefore, the conventional cleaning process using a solvent and ultrasonic wave may compromise the quality and reliability of the device.

As shown in FIG. 2f, the sacrificial part (12) is etched by dry etching.

In this example step (f) of etching can be done by any effective dry etch to remove the sacrificial part. Because of the existence of the protruded structure, the metal particles re-sputtered during the step (e) mainly lands on the masking layer. Much less of the metal particles would remain in the space between the electrodes.

In some embodiments, the dry etching in step (f) comprises a continuous ion-beam etching from step (e). In other words, in some embodiments, the ion-beam etching initiated in step (e) continues in step (f), thereby constituting the dry etching process for at least a portion of the sacrificial part in step (f).

Thus, the resistance at the smallest distance (d1) between the plurality of electrodes (15), as indicated in FIG. 1g, can be at least 1e8 ohm.

After the sacrificial part is etched, the dielectric layer (11) comprises regions corresponding to the removed sacrificial part.

In some embodiments, the sacrificial part (12) is further etched by non-ion-beam dry etching, e.g., reactive ion etching. The reactive ion etching can selectively remove the material of the sacrificial part (12) without damaging the electrodes (15). In some embodiments, the electrodes act as hard mask for reactive ion etching the sacrificial part (12).

Because of the non-ion-beam dry etch, the density of metal particles close to the edge of the electrode is further reduced thus increasing the resistance between the electrodes.

A stack (24) comprising the substrate (10), the dielectric layer (11), a patterned conductive layer (13), e.g., the electrodes (15), and a patterned masking layer (14) is formed after the step (f) and before step (g).

In some embodiments, as a last step, the masking layer (14) is removed by (e.g., conventional) etching methods. A stack (25) comprising the substrate (10), the dielectric layer (11) and a patterned conductive layer (13), e.g., the electrodes (15) is formed after step (f).

In some other embodiments, the masking layer (14) can be removed before the further non-ion-beam dry etching, e.g., reactive ion etching, by etching methods.

As indicated in FIG. 2g, the distance (d1) between the electrodes is provided (e.g., defined) in the step (d) of patterning the masking layer (14) to expose at least part of the conductive layer (13) above the sacrificial part (12).

The pitch (d3), as indicated in FIG. 2h, is provided (e.g., defined) after step (e) of etching the conductive layer (13) by ion-beam thereby forming the plurality of electrodes (15).

The distance (d1) between the electrodes may be the smallest distance between any two electrodes of the plurality of electrodes. The resistance of the two electrodes which has the smallest distance can be less than 1e8 ohm after dry etching step (f).

In some embodiment, the dry etching in step (f) comprises a further step of non-ion-beam etching, e.g., reactive ion etching.

The dry etching can have an etching rate ranging from 10 nm/min to 1000 nm/min depending on the material of the sacrificial part (12), the etching gas, and the etching parameters. The dry etching can also be controlled by adjusting the duration and the area of exposure. The dry etching removes at least a portion of the sacrificial part (12) to form a cavity (17) that has a depth ranging from 10 nm to 200 nm.

After the sacrificial part is etched, the dielectric layer (11) comprises recessed regions corresponding to the removed sacrificial part.

A stack (24) comprising the substrate (10), the dielectric layer (11) with recessed regions, a patterned conductive layer (13), e.g., the electrodes (15), and a patterned masking layer (14) is formed after the step (f) and before step (g).

As a last step, the masking layer (14) is removed by (e.g., conventional) etching methods. A stack (25) comprising the substrate (10), the dielectric layer (11) with recessed region and a patterned conductive layer (13), e.g., the electrodes (15) is formed after step (f).

As indicated in FIG. 2g, the distance (d1) between the electrodes is provided (e.g., defined) in the step (d) of patterning the masking layer (14) to expose at least part of the conductive layer (13) above the sacrificial part (12). In an embodiment, the distance (d2) of the recessed region in the dielectric layer between the electrodes is substantively equal to the distance (d1) between the electrodes. In an embodiment, the distance (d2) of the recessed region in the dielectric layer between the electrodes is within 10% margin of the distance (d1) between the electrodes.

The pitch (d3), as indicated in FIG. 2h, is provided (e.g., defined) after step (e) of etching the conductive layer (13) by ion-beam thereby forming the plurality of electrodes (15).

The distance (d1) between the electrodes can be the smallest distance between any two electrodes of the plurality of electrodes. The resistance of the two electrodes which has the smallest distance can be less than 1e8 ohm after dry etching step (f).

To measure the resistance between the electrodes, the probes of the multimeter or the ohmmeter can be connected to the two electrodes whose resistance is to be measured.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A method for manufacturing a plurality of electrodes wherein the electrodes comprise noble metal, the method comprising:
providing a substrate having a dielectric layer in physical contact therewith,
wherein the dielectric layer comprises at least a sacrificial part, wherein the sacrificial part comprises a top surface;
forming a conductive layer on the dielectric layer and in physical contact with the top surface of the sacrificial part, wherein the conductive layer comprises the noble metal;
providing a masking layer over the conductive layer;
patterning the masking layer to expose at least the conductive layer above the sacrificial part thereby defining a plurality of electrodes wherein the smallest distance between the plurality of electrodes is not larger than 500 nm;
etching the conductive layer by ion-beam thereby forming the plurality of electrodes comprising the noble metal;
etching at least a portion of the sacrificial part by dry etching such that resistance at the smallest distance between the plurality of electrodes is at least 1e8 ohm; and
removing the masking layer.

2. The method according to claim 1, wherein the sacrificial part comprises a protruded structure of the dielectric layer.

3. The method according to claim 2, wherein etching at least a portion of the sacrificial part by dry etching comprises ion-beam etching.

4. The method according to claim 2, wherein etching at least a portion of the sacrificial part by dry etching comprises reactive ion etching.

5. The method according to claim 1, wherein a top surface of the dielectric layer is planar and the top surface of the sacrificial part is coplanar with a surrounding top surface of the dielectric layer, wherein etching at least a portion of the sacrificial part by dry etching comprises reactive ion etching.

6. The method according to claim 1, wherein the noble metal is selected from at least one of platinum (Pt), gold (Au), or silver (Ag).

7. The method according to claim 1, wherein the dielectric layer comprises SiO₂ or SiₓN_{y}.

8. The method according to claim 1, wherein the masking layer comprises SiO₂, SiₓN_{y}, Ti, TiN, or DLC.

9. The method according to claim 1, wherein etching at least a portion of the sacrificial part by dry etching is performed such that a recessed region in the dielectric layer is obtained between the plurality of electrodes.

10. The method according to claim 1, wherein the sacrificial part is recessed for at least 20 nm with regard to the top surface after the step of etching at least a portion of the sacrificial part by dry etching such that the resistance at the smallest distance between the plurality of electrodes is at least 1e8 ohm.

11. An electrode array comprising a plurality of electrodes, wherein the plurality of electrodes comprise noble metal, wherein the plurality of electrodes are manufactured via a method comprising:
providing a substrate having a dielectric layer in physical contact therewith, wherein the dielectric layer comprises at least a sacrificial part, wherein the sacrificial part comprises a top surface;
forming a conductive layer on the dielectric layer and in physical contact with the top surface of the sacrificial part, wherein the conductive layer comprises the noble metal;
providing a masking layer over the conductive layer;
patterning the masking layer to expose at least the conductive layer above the sacrificial part thereby defining a plurality of electrodes wherein the smallest distance between the plurality of electrodes is not larger than 500 nm;
etching the conductive layer by ion-beam thereby forming the plurality of electrodes comprising the noble metal;
etching at least a portion of the sacrificial part by dry etching such that resistance at the smallest distance between the plurality of electrodes is at least 1e8 ohm; and
removing the masking layer.

12. The electrode array according to claim 11, wherein the array has a pitch distance of not larger than 500 nm.

13. The electrode array according to claim 11, wherein the array comprises at least a thousand electrodes or a million electrodes.

14. The electrode array according to claim 11, wherein the electrodes of the electrode array comprise platinum.

15. An electroactive device for bio-material processing, the electroactive device comprising:
an electrode array comprising a plurality of electrodes, wherein the plurality of electrodes comprise noble metal and wherein the plurality of electrodes are manufactured via a method comprising:
providing a substrate having a dielectric layer in physical contact therewith, wherein the dielectric layer comprises at least a sacrificial part, wherein the sacrificial part comprises a top surface;
forming a conductive layer on the dielectric layer and in physical contact with the top surface of the sacrificial part, wherein the conductive layer comprises the noble metal;
providing a masking layer over the conductive layer;
patterning the masking layer to expose at least the conductive layer above the sacrificial part thereby defining a plurality of electrodes wherein the smallest distance between the plurality of electrodes is not larger than 500 nm;
etching the conductive layer by ion-beam thereby forming the plurality of electrodes comprising the noble metal;
etching at least a portion of the sacrificial part by dry etching such that resistance at the smallest distance between the plurality of electrodes is at least 1e8 ohm; and
removing the masking layer; and
a reservoir for containing bio-material in an electrolyte during operation, wherein the electrode array is arranged such that the electrode array is exposed to the electrolyte during the operation of the electroactive device.

16. The electroactive device according to claim 15, wherein the electrode array has a pitch distance not larger than 500 nm.

17. The electroactive device according to claim 15, wherein the electrode array comprises at least a thousand electrodes or a million electrodes.

18. The electroactive device according to claim 15, wherein the plurality of electrodes of the electrode array comprise platinum.

19. The electroactive device according to claim 15, wherein the dielectric layer comprises SiO₂ or SiₓN_{y}.

20. The electroactive device according to claim 15, wherein the masking layer comprises SiO₂, SiₓN_{y}, Ti, TiN, or DLC.
